(19) United States
(12) Patent Application Publication
(10) Pub. No.: US 2008/0182835 A1
(43) Pub. Date: Jul. 31, 2008

(54) METHOD OF USING A COBALT-AMINE BASED METAL COMPLEX AS AN ANTIVIRAL COMPOUND AND A METHOD FOR THE PREPARATION OF FUNCTIONALIZED ANALOGS THEREOF

(75) Inventors: Eddie L. Chang, Silver Spring, MD (US); James B. Delehanty, Washington, DC (US); Dzung C. Thach, Annandale, VA (US); Jason Bongard, Alexandria, VA (US); Andy Knight, New Orleans, LA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignee: The Government of the US, as represented by the Secretary of the Navy, Washington, DC (US)

(21) Appl. No.: 11/934,424

(22) Filed: Nov. 2, 2007

Related U.S. Application Data
(60) Provisional application No. 60/865,690, filed on Nov. 14, 2006.

(51) Int. Cl. A61K 31/555; C12N 5/06; C07H 21/04; A61P 31/12

(52) U.S. Cl. 514/184; 435/375; 536/23.1

(57) ABSTRACT

The present invention is generally directed to a method of prophylaxis against viral infection of a cell or subject or a method of treating a subject infected with a virus including administering an antiviral composition having the general Structure III,

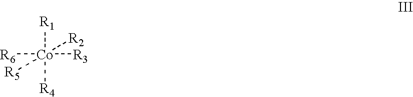

wherein each of R₁, R₂, R₃, R₄, R₅ and R₆ is the same or different and includes an N-based ligand donor atom selected from the group consisting of ammonia, primary amine or secondary amine, or salt thereof. The present invention is also generally directed to a method of preparing an antiviral agent including providing a cobalt pentammine salt having a non-amine coordination site and mono-substituting the non-amine coordination site with a functional group incorporating a strong coordinator atom to cobalt to form a CoHex structure of Structure III, in which R₁ incorporates the functional group having the strong coordinator atom coordinated with the cobalt atom, or a salt thereof.

### FIELD OF THE INVENTION

The present invention is generally directed to the use of a cobalt-amine based metal complex as an antiviral compound and a method for the preparation thereof.

### BACKGROUND OF THE INVENTION

Unlike antibiotics, there are significantly fewer antiviral drugs available. For example, for influenza there are only four: amantadine, rimantadine, oseltamivir (Tamiflu), zanamivir. These four drugs can be divided into two categories, the adamantane derivatives (amantadine, rimantadine) and the neuraminidase inhibitors (oseltamivir, zanamivir), on the basis of their chemical properties and activities against influenza viruses. Adamantanes inhibit influenza propagation by blocking the viral M2 protein ion channel, which prevents fusion of the virus and host-cell membranes and release of viral RNA into the cytoplasm of infected cells. Neuraminidase inhibitors, on the other hand, block the process of release of influenza virus from infected cells and, thereby, inhibit virus transmission to the neighboring cells. Another example of an antiviral drug is cidofovir, which has been found to be effective for treatment against cytomegalovirus, a virus that puts babies and people with HIV at risk. As with antibiotics, these antiviral agents exhibit problems with either resistance or toxicity, thereby limiting options for treatment of viral infection. Thus, there is some urgency in the development of new classes of antiviral drugs.

Certain metal-ion based antiviral complexes, such as the CTC series of cobalt(III)-based compounds has been shown to possess anti-inflammatory and antiviral activity. Structure I is a general formula for a CTC complex.

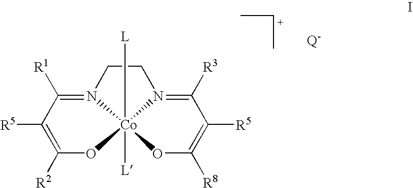

As illustrated, CTC complexes are generally complex chelate structures, with interconnected cobalt coordination sites, leaving only axial coordination cites accessible for activity. In particular, these axial positions contain labile, or easily altered or broken down, axial ligands. For example, in CTC-96 (also known as DOXOVIR, commercially available from Redox Pharm. Corp.), the most effective of the series, the labile axial ligands are 2-methyl-imidazoles.

Several CTC complexes have moderate in vitro and in vivo activity against herpes simplex virus types 1 and 2, varicella-zoster virus, cytomegalovirus, and Epstein-Barr virus. While the therapeutic activity of the CTC series has been known for several years, the mechanisms and the stage of the virus life cycle at which many of these compounds are effective are only beginning to be understood. For example, several CTC compounds can bind strongly to, and inhibit, Sp1, a DNA binding Zn finger protein, implying that the locus of inhibition may be through the group of metalloproteins that depend on the Zn finger motif. More recently, alternative mechanisms of inhibition have been postulated based on the inhibition of virus-mediated cell fusion.

Another type of Cobalt(III)-based compounds, based on the macrocyclic cyclen chelator, have been shown to bind very tightly to DNA/RNA, to hydrolyze the phosphodiester bonds of the nucleotides, and to inhibit protein translation in cell-free translation lysates. These cyclen complexes were of particular interest because they have only four out of six possible coordination sites on the Co(III) ion bound, leaving two cis-equatorial positions open for hydrolysis by the complex. For example, structure II below is a CoCyclen (or 1,4,7,10 tetraazacyclododecane) molecule illustrating the two cis-equatorial positions open for hydrolysis by the complex.

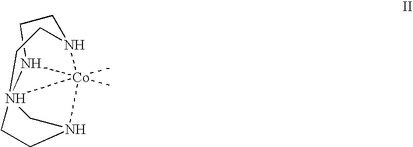

These cyclen complexes have been used for mechanistic studies of phosphodiester cleavage for both its efficient hydrolysis rates and kinetic inertness. That is, the complexes promote fast hydrolysis of the phosphodiester bond but are kinetically “slow” in letting go of the hydrolyzed phosphate. The kinetic inertness of the Co(III) may be overcome (i.e., at elevated temperatures) but, for gene-silencing, this property has the added advantage of disruption of gene function, particularly disruption of protein translation. However, even potent hydrolytic catalysts take much longer to degrade nucleic acids than enzymes.

Other antiviral approaches include “antisense” technology which includes the synthesis of oligodeoxynucleotides (ODN's) that bind to their complementary sequences on the mRNA, thereby blocking translation and inhibiting the production of the target protein. However, binding to the RNA is often not stable. Ribosomes can effectively compete with the oligonucleotides to bind with the RNA and consequently ensure continuous production of the target protein. The competitive edge of ribosomes is facilitated by their intrinsic “unwindase” activity, which allows them to read tangled messages, thus overcoming the effect of the antisense ODN.

One solution to this “unwindase” activity relies on the ability of the antisense oligonucleotides to employ the enzyme RNase H. RNase H recognizes the DNA:RNA duplex and acts as a DNA dependent RNA hydrolysis catalyst. This degrades the RNA leaving the antisense ODN free to bind to other mRNA molecules, where the RNA hydrolysis cycle is repeated. However, RNase H itself poses a drawback because DNA:RNA duplexes as short as 5 base pairs may be cleaved by RNase H, leading to poor specificity of the antisense ODN's.

The lack of stability of the antisense ODN is also a drawback for anitsense technology. Conventional ODN's are prone to nuclease degradation inside the cell. Modified ODN's have been synthesized with different backbones to improve their stability but they either fail to recruit RNase H or exhibit non-sequence specificity. A classical example of synthesized stable antisense oligos is S-DNA (phosphorothioate) oligos that possess enhanced stability but exhibit low sequence specificity because of their weak binding. In addition, they promiscuously bind cellular protein molecules thus reducing their effectiveness as antisense agents.

Alternatively, peptide nucleic acids, in which the backbone consists of N-(2-aminoethyl)-glycine units linked by peptide bonds instead of a sugar and phosphate groups, have gained considerable importance by virtue of their being nuclease resistant and their ability to form stable complexes with nucleic acids. More recently, morpholino oligos have also been reported to afford high efficacy, specificity, and resistance to nucleases. However, they fail to recruit RNase H relying only on the binding specificity of the oligo.

Another approach includes RNA interference (RNAi) therapeutics. The major difficulties with RNAi technology lie with the lack of a reliable method of targeting and delivery of double-stranded RNA (dsRNA). Issues, such as activation of the cells' antiviral defense mechanisms by long strand dsRNA, identification of a viable target region, and uncontrolled global changes in gene expression of cells when dsRNA strands are introduced into the cells, complicate any potential RNAi applications using short, interfering RNA (siRNA). While activation of cellular antiviral mechanisms may be desirable for antiviral applications, many viruses can shut down the defenses once entry is achieved. This natural viral defense strategy can be an impediment for siRNA therapeutics.

Since only short RNA strands can be used for siRNA, specificity of the target also can be an issue. Additionally, determining which sequences will work for siRNA still remains a problem to be solved for each target gene. Therefore, multiple regions are generally screened for each target. The screening requires using either synthetic RNAs, which are expensive, or with cloned DNA sequences, which is time consuming. Furthermore, the activity of the siRNA is not well understood at present—not all sequences will work and it is still a hit-and-miss proposition to find an active sequence.

### BRIEF SUMMARY OF THE INVENTION

The CoHex complex of the present invention exhibits a potent inhibition of virus replication without possessing free cobalt(III) coordination sites and without hydrolyzing oligonucleotides.

An embodiment of the present invention includes a method of prophylaxis against viral infection of a cell including administering to a cell an antiviral composition having the structure of Structure III,

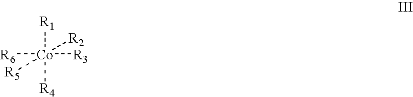

wherein each of R₁, R₂, R₃, R₄, R₅ and R₆ is the same or different and includes a N-based ligand donor atom selected from the group consisting of ammonia, primary amine or secondary amine, or salt thereof, so as to thereby provide prophylaxis against infection of the cell by a virus.

An alternative embodiment of the present invention includes a method of treating a subject infected with a virus including administering to the subject an antiviral composition comprising an antiviral effective amount of a compound having the structure of Structure III, above, wherein each of R₁, R₂, R₃, R₄, R₅ and R₆ is the same or different and includes a N-based ligand donor atom selected from the group consisting of ammonia, primary amine or secondary amine, or salt thereof, so as to thereby treat the subject infected by the virus.

An alternative embodiment of the present invention includes a method of prophylaxis against viral infection of a subject including administering to a cell an antiviral composition having the structure of Structure III, above, wherein each of R₁, R₂, R₃, R₄, R₅ and R₆ is the same or different and includes a N-based ligand donor atom selected from the group consisting of ammonia, primary amine or secondary amine, or salt thereof, so as to thereby provide prophylaxis against infection of the subject by the virus.

An alternative embodiment of the present invention includes a method of preparing an antiviral agent including providing a cobalt pentammine salt having a non-ammine coordination site and mono-substituting the non-ammine coordination site with a functional group incorporating a strong coordinator atom to cobalt to form a CoHex structure of Structure III, above, where each of R₂, R₃, R₄, R₅ and R₆ is the same or different and includes a N-based ligand donor atom selected from the group consisting of ammonia, primary amine or secondary amine and R₁ incorporates the functional group having the strong coordinator atom coordinated with the cobalt atom, or a salt thereof.

The foregoing and other features and advantages of the present invention will be apparent from the following, more particular description of a preferred embodiment of the invention, as illustrated in the accompanying drawings.

### BRIEF DESCRIPTION OF THE DRAWINGS

FIG. 1 is a graph plotting percentage of cell viability as a function of the concentration of a CoHex complex of the present invention as compared to a conventional antiviral compound.

FIG. 2 is a western blot exhibiting inhibition of luciferase protein translation in vitro by a CoHex complex of the present invention.

FIG. 3A is a graph plotting virus plaque counts in log plaque formation units as a function of the concentration of a CoHex complex of the present invention. FIG. 3B is a graph plotting a degree of inhibition of plaque formation as a function of concentration of the CoHex complex of the present invention based on the results illustrated in FIG. 3A. FIG. 3C is a graph plotting the fraction of cell survival of BHK cells during viral infection as a function of the concentration of a CoHex complex of the present invention relative to virus infected cells that received no CoHex complex treatment.

FIG. 4 is a graph plotting virus plaque counts in log plaque formation units per percent cell viability as a function of the concentration of a CoHex complex of the present invention.

FIG. 5 is a graph plotting percent cell viability as a function of the concentration of a CoHex complex of the present invention.

FIG. 6A is a micrograph illustrating healthy BHK cells in the absence of a virus and a CoHex complex of the present invention. FIG. 6B is a micrograph of BHK cells infected with a virus but no CoHex complex of the present invention. FIGS. 6C-6H are micrographs of BHK cells infected with a virus and concentrations of 0.15 mM, 0.3 mM, 0.6 mM, 1.2 mM 2.5 mM and 5 mM, respectively, of a CoHex complex of the present invention.

FIG. 7A is a raw data plot of simultaneous analysis of Sindbis virus protein synthesis during SV infection in the absence of a CoHex complex of the present invention. FIG. 7B is a raw data plot of simultaneous analysis of Sindbis virus protein synthesis during Sindbis virus infection in the presence of a CoHex complex of the present invention. FIG. 7C is representative flow cytometry data for the viability of Sindbis virus-infected BHK cells in the absence of a CoHex complex of the present invention. FIG. 7D is representative flow cytometry data for the viability of Sindbis virus-infected BHK cells in the presence of a CoHex complex of the present invention.

FIG. 8A is a chart illustrating the dose-dependent increase in cell viability in Sindbis virus-infected cells (solid square) as a function of the concentration of a CoHex complex of the present invention relative to uninfected cells (open circle). FIG. 8B is a chart illustrating the dose-dependent inhibition of EGFP expression in Sindbis virus-infected cells (solid square) as a function of the concentration of a CoHex complex of the present invention compared to uninfected cells (open circle).

FIG. 9 is a chart illustrating the dose-dependent increase in cell viability in Sindbis virus-infected cells as a function of the concentration of a CoHex complex of the present invention when the CoHex complex is administered at various times.

FIG. 10 is a chart illustrating the dose-dependent inhibition of EGFP expression in Sindbis virus-infected cells as a function of the concentration of a CoHex complex of the present invention when the CoHex complex is administered at various times.

FIG. 11 is a graph illustrating the dose-dependent increase in cell viability in Adenovirus-infected cells (right bar) as a function of the concentration of a CoHex complex of the present invention relative to uninfected cells (right bar).

FIG. 12 is graph comparing the increase in cell viability in Sindbis-infected cells (left bar) and Adenovirus-infected cells (right bar) normalized to respective infected control cells as a function of the concentration of a CoHex complex of the present invention.

FIG. 13 is graph comparing the increase in cell viability in Sindbis-infected cells (left bar) and Adenovirus-infected cells (right bar) normalized to respective uninfected control cells as a function of the concentration of a CoHex complex of the present invention.

FIG. 14 is a schematic illustrating a method for synthesizing an example of a functionalized CoHex complex of the present invention.

### DETAILED DESCRIPTION OF THE INVENTION

Preferred embodiments of the present invention are now described with reference to the Figures. While specific details of the preferred embodiments are discussed, it should be understood that this is done for illustrative purposes only. A person skilled in the relevant art will recognize that other configurations and arrangements can be used without departing from the spirit and scope of the invention. It will also be apparent to a person skilled in the relevant art that this invention can also be employed in other applications.

The identification of new antiviral drugs is a challenging endeavor that is often built upon the balance between potent antiviral activity and minimal toxicity against host tissues. The present invention is generally directed to positively charged cobalt(III) complexes or salts thereof in octahedral ligand environments in which the ligands have strong coordinator atoms to Cobalt (i.e., CoHex complex), for example, where the ligand donor atoms are nitrogen (N)-based and/or interact either electrostatically or via hydrogen bonding, particularly ammonia, primary amines or secondary amines. The present invention may be demonstrated by the general formula of structure III, wherein R₁ through R₆ is the same or different and includes N-based ligand donor atoms of ammonia, primary amines or secondary amines.

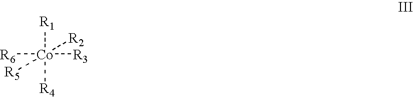

A few examples of CoHex complexes of the present invention are found below. Structure IV has all six positions coordinated with a nitrogen atom of ammonia ligands, i.e., Co(NH₃)₆. Structure V is similar to the CoCyclen of Structure II, however, with all six positions coordinated with a N atom and with ammonia ligands at the cis-equatorial positions, rather than having these positions open for hydrolysis. In Structure V, some of the N-based ligand donors are chelating ligands. The CoHex complexes of the present invention are highly positively charged compounds. For example, the net charge for the complex of Structure IV of 3⁺ at neutral pH. Preferably, they are prepared so as to be administered to a cell or subject as an acceptable salt.

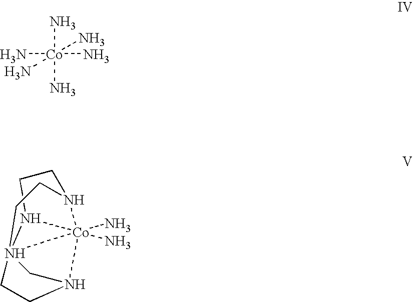

Neither UV-VIS nor NMR shows evidence that Co(NH₃)₆ (i.e., Structure IV) has an ability to exchange its ammonia groups with free histidine, even at a ratio 100:1 histidine to Co(NH₃)₆ (results not shown). In other words, Co(NH₃)₆ does not appear to have labile ligands as does the conventional CTC complexes. Surprisingly though, the CoHex complexes of the present invention provide potent antiviral activity and relatively minimal toxicity against host tissues.

### Preparation of Cobalt Complexes

### EXAMPLE 1

While Co(NH₃)₆ is available commercially, the synthesis of its chlorine salt is fairly straight forward and using easily available reagents, for example using air to oxidize Co(II) to Co(III) according to the following formula.

CoCl₂+4NH₄Cl+20NH₃+O₂→4[Co(NH₃)₆]Cl₃+2H₂O

The example discussed below is for Co(NH₃)₆, but one skilled in the art can appreciate that other CoHex complexes of the present invention may be commercially available or synthesized using similar methods. To prepare this Co(NH₃)₆ example, or more specifically the chlorine salt thereof, 9.6 g of CoCl₂.6H₂O (0.06 mol) and 6.4 g of NH₄Cl (0.12 mol) were added to 40 mL of water in a 250 mL Erlenmeyer flask with a side arm. The mixture was shaken until most of the salts are dissolved. Then, 1 g of fresh activated decolorizing charcoal and 20 mL concentrated ammonia were added. The flask was next connected to the aspirator or vacuum line and air drawn through the mixture until the red solution became yellowish brown (about 2-3 hours). The air inlet tube was of fairly large bore (about 10 mm) to prevent clogging with the precipitated Co(NH₃)₆³⁺ salt.

The crystals and charcoal were filtered on a Büichner funnel and then a solution of 6 mL of concentrated hydrochloric acid (HCl) in 75 mL of water was added, the mixture heated on a hot plate to effect complete solution and filtered while hot. Crystallization of the CoHex chloride was done by cooling to 0° C. and then slowly adding 15 mL of concentrated HCl. The crystals were washed with 60% and then with 95% ethanol and dried at 80-100° C.

### Cytotoxicity of CoHex

### EXAMPLE 2

The cytotoxicity of Co(NH₃)₆ (Structure IV) was assessed by monitoring its ability to inhibit proliferation of baby hamster kidney (BHK) cells. BHK cells were cultured as exponentially growing subconfluent monolayers in complete growth medium, particularly Dulbecco's Modified Eagle's Medium (DMEM) supplemented with 1% (v/v) antibiotic/antimycotic agent and 10% (v/v) heat inactivated fetal bovine serum (FBS). Cells were grown in either T25 or T75 flasks (Acton, Mass.) and incubated at 37° C. under 5% CO₂ atmosphere. A subculture was performed every 3-4 days.

The in vitro toxicity of Co(NH₃)₆ was determined using the CellTiter96® Proliferation Assay (Promega, Madison, Wis.). This quantitative calorimetric assay is based upon the enzymatic conversion of a tetrazolium salt substrate into a blue formazan product with a maximum absorbance at 570 nm. When incubated with this substrate, only viable cells convert the substrate into a blue product while nonviable cells do not. At the assay endpoint, the absorbance at 570 nm is directly proportional to the number of viable cells. To assess compound toxicity, BHK cells were seeded into the wells of a 96-well tissue culture microtiter plate (2×10⁴ cells/well) and cultured overnight at 37° C. in a humidified atmosphere containing 5% CO₂. The next day, Co(NH₃)₆ was diluted into tissue culture media (at final concentrations ranging from 0 to 5 mM) and incubated with the cells for 72 hrs. Triplicate wells were included for each concentration. To control for any contaminating absorbance due to the compounds, control wells containing no cells were included at each compound concentration (“no cell” wells). At the end of the 72 hour culture period, 15 μL of tetrazolium substrate was added to each well and the plate was returned to the incubator for 4 hours to allow viable cells to convert the substrate into the formazan product. At the end of the 4 hour incubation period, 100 μL of solubilization solution was added to each well and the plate was subsequently incubated overnight to completely solubilize the contents of each well to a homogeneous color. The absorbance within each of the “no cell” wells was subtracted from the absorbance measured in the corresponding wells containing cells for each concentration of Co(NH₃)₆.

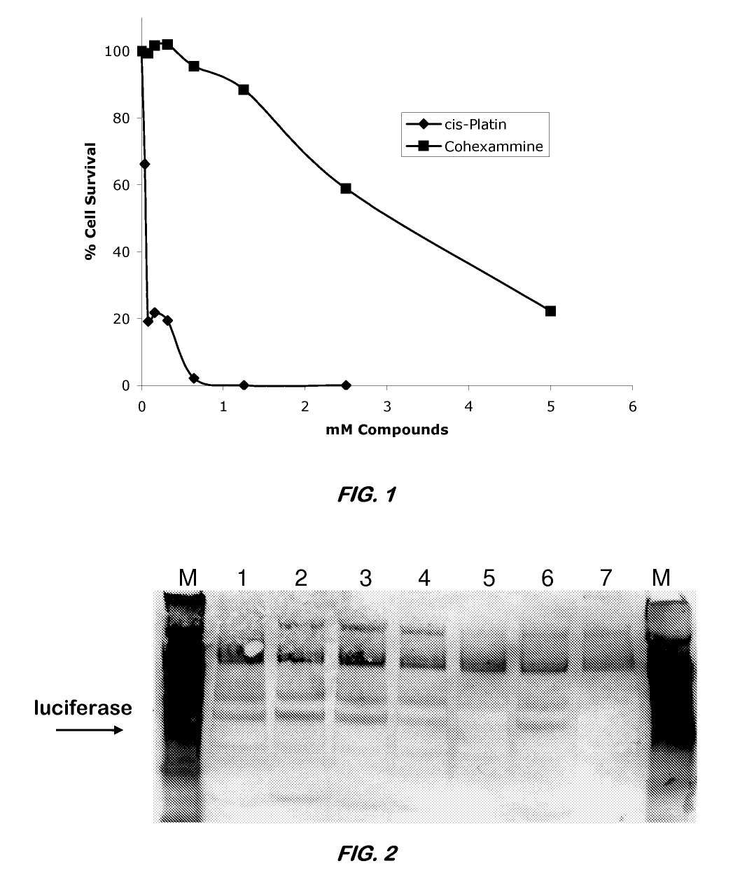

FIG. 1 illustrates cell viability with increasing concentrations of Co(NH₃)₆ as compared with a conventional cis-Platin complex. cis-Platin is an FDA approved and commercially available medicament used as an anti-cancer agent. As shown in FIG. 1, Co(NH₃)₆ elicited a dose-dependent inhibition of cellular proliferation in which concentrations below 2.5 mM were only mildly toxic, with no significant toxicity at concentrations below 0.3 mM. At concentrations above 2.5 mM, however, Co(NH₃)₆ increasingly inhibited cellular proliferation. At 2.5 mM Co(NH₃)₆ mediated about 80% cell viability, while achieving a maximum toxicity at 5 mM, the highest concentration tested. Also as demonstrated by FIG. 1, Co(NH₃)₆ is much less toxic than that of the FDA approved anti-cancer agent, cis-Platin. Lower toxicity makes a CoHex complex of the present invention a good source as a therapeutic.

From FIG. 1, a 50% cytotoxicity concentration (CC₅₀) for CoHex, i.e., the concentration at which approximately 50% cell death occurs due to the administration of CoHex, was determined to be about 3.2 mM.

### Activity of the CoHex Complex in Preventing Translation

The highly positively charged CoHex complex has built-in “stickiness” for highly-dense, negatively charged polyelectrolytes, such as DNA/RNA, such that it is tightly bound and does not easily release the nucleotides. This tight binding encourages blocking of translation and is similar in concept to “antisense” and RNAi technologies, but does not include some of the limitations inherent to the other approaches. In particular, because the CoHex complex itself binds strongly to nucleotides via static charges and hydrogen bonding, the tight binding overcomes any attempts by the cellular machinery to “unwind” the hybridization site.

CoHex complexes have been previously studied for its ability to “condense” double stranded DNA into toroidal-like superstructures under low salt conditions. Nonetheless, without the hydrolytic open binding sites, the CoHex complex of the present invention is not a potent gene-silencing agent. Surprisingly, however, a hydrolytic function is not necessary for suppressing protein translation. The CoHex complex has the characteristic of binding very tightly to nucleotides and has demonstrated that such a tight ionic-binding property is sufficient for the inhibition of protein expression and thus antiviral activities.

CoHex complexes, with their high positively charged density, are ideal for binding nucleotides and other polyelectrolytes. Although Cobalt(III) is not stable, by itself, in aqueous solutions, it can be stabilized by coordinating with donor atoms (preferably N-based ligands and chelators) that make strong contributions to the ligand field. While it is not clear why CoHex complexes are particularly useful for antiviral activity, the kinetic inertness, i.e., the inability of the close and tightly bound N atoms to easily disassociate from the Cobalt atom, may play a role in the particularly good antiviral activity of CoHex complexes. The role of close and tightly bound N atoms in CoHex's antiviral activity is particularly surprising, since it is the labile axial ligands, as in Structure II, and/or the availability of coordinating positions, as in Structure I, that is the apparent mode of antiviral activity of the conventional antiviral compounds.

Suppressing protein translation is to inhibit the ability of either DNA or RNA to act as templates for transcription and translation, respectively. This is done by blocking the site of transcription/translation or by hydrolyzing the DNA/RNA template. The CoHex complex of the present invention does not hydrolyze nucleotides, but nonetheless demonstrates potent protein-inhibition and dose-dependent antiviral properties by blocking the site of transcription/translation. For purposes of gene knock-out or antiviral applications, it is not necessary to fully digest a DNA or RNA strand to deactivate an organism or a gene, as long as the critical sites remain permanently blocked. The strong affinity of the CoHex complexes of the present invention for the bases and the phosphate groups of the nucleotides, therefore, is a critical advantage to the general goal of deactivating genetic materials. As such, the CoHex complex of the present invention can function as non-specific gene silencing compounds or general antiviral applications.

### EXAMPLE 3

CoHex is capable of preventing translation of a messenger RNA (mRNA) in vitro. The ability Co(NH₃)₆ to inhibit translation of an mRNA luciferase template was assessed using the Rabbit Reticulocyte Lysate Translation System (Promega) according to the manufacturer's instructions. In the example discussed below, Co(NH₃)₆ in various concentrations was incubated with the mRNA for 10 minutes before the addition of the mRNA template to the translation lysate. Translated luciferase protein was run on a 10% SDSPAGE gel for 45 min at 130 V and detected by Western blot on a PVDF membrane. Proteins were detected using a streptavidinalkaline phosphatase conjugate and Western Blue substrate according to the instructions in the Transcend Non-Radioactive Translation Detection System (Promega). The results are provided in FIG. 2. In FIG. 2, Lane 1 represents the incubation of 0.01 mM Co(NH₃)₆. Lane 2 represents the incubation of 0.02 mM Co(NH₃)₆. Lane 3 represents the incubation of 0.05 mM Co(NH₃)₆. Lane 4 represents the incubation of 0.1 mM Co(NH₃)₆. Lane 5 represents the incubation of 0.2 mM Co(NH₃)₆. Lane 6 is a positive control in which no Co(NH₃)₆ is added. Lane 7 is a negative control in which no translation occurs. The lanes marked M are molecular weight marker lanes. As shown in FIG. 2, Co(NH₃)₆ blocks the RNA translation of luciferase protein at around concentrations of about 0.1 to 0.2 mM Co(NH₃)₆. Further, when compared to the same test using a conventional CoCyclen structure similar to Structure II rather than Co(NH₃)₆ (data not provided), Co(NH₃)₆ inhibits protein translation about an order of magnitude (or about 10 times) more than the conventional CoCyclen structure.

### Antiviral Activity of the CoHex Complexes—Sindbis Virus

### EXAMPLE 4

In order to provide an example of antiviral activity, an in vitro model of a positive single stranded RNA (+ssRNA) virus, recombinant Sindbis virus (633-EGFP strain), infection was used. In this construct, the gene encoding enhanced green fluorescent protein (EGFP) is placed under the control of the identical promoter sequence found upstream of the viral structural proteins. Thus, upon replication, virally-infected cells produce soluble EGFP at levels that are proportional to the amount of virus. The virus seed stock was expanded on BHK cells under serum free conditions.

BHK cells were grown in T-150 tissue culture flasks (Acton, Mass.) until 90% confluent. Subsequently, the cells were washed twice with Dulbecco's Phosphate Buffered Saline (PBS) and infected with the recombinant Sindbis virus at a multiplicity of infection of five plaque forming units (pfu) per cell in 2 ml of virus production serum free medium (VP-SFM). After 1 hour of incubation at 37° C., with rocking of flasks every ten minutes, an additional 13 ml of VP-SFM media was added to bring the volume to 15 ml. The next day, the cells were observed under light microscopy for signs of cytopathic effects and the presence of EGFP expression in infected cells was confirmed via fluorescence microscopy. Cellular debris was centrifuged and the supernatant was collected, aliquoted, and stored at −80° C. The majority of cells (greater than 70%) were positive for EGFP fluorescence.

### Plaque Formation Assay

### EXAMPLE 5

BHK cells (1×10⁵ cells) were seeded to the wells of a 24-well plate and grown overnight to a confluent monolayer. The next day, the cells were infected with the Sindbis virus at a ratio of about five plaque forming units (pfu) per cell in DMEM containing 2% FBS in the absence or the presence of increasing concentrations of Co(NH₃)₆. Infection was allowed to proceed for 48 hours, at which time the supernatants were collected and stored at −80° C. until further use.

For quantization of viral infection and replication, the sampled supernatants were thawed and serially diluted in 1% FBS DMEM. The diluted virus was used to infect monolayers of BHK cells grown in 6-well clustered plates. An aliquot (200 μL) of virus from each selected dilution was incubated with BHK monolayers for 1 hr at 37° C. with rocking of plates every 10 min to prevent drying of the monolayers. After 1 hr, the cells were overlayed with warm melted 1.2% Bacto agar in water mixed with an equal volume of 2× Minimum Essential Medium (MEM). The overlay agar was allowed to solidify at room temperature. Then the plates were incubated at 37° C. for 48 hr to allow plaque formation. Two days later, the cells were stained with neutral red. The exact staining solution per well was 0.5 ml of 2×MEM, 0.5 ml distilled water, 0.11 ml of 0.33% neutral red solution (i.e., 3.3 g/L in PBS). After 1-2 hrs of staining, the remaining staining solution was aspirated off and plaques were observed as clear foci within the cell monolayer. Via this method the amount of virus in the cells (i.e., plaque counts) can be ascertained by visual counting the number of plaque formation units (pfu) per well and multiplying by the dilution factor to determine the concentration of pfu in the original supernatant.

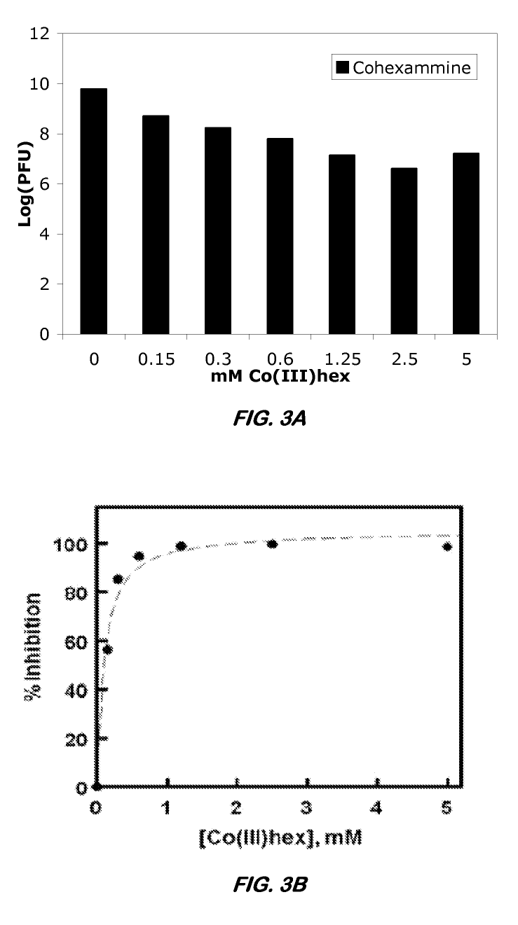

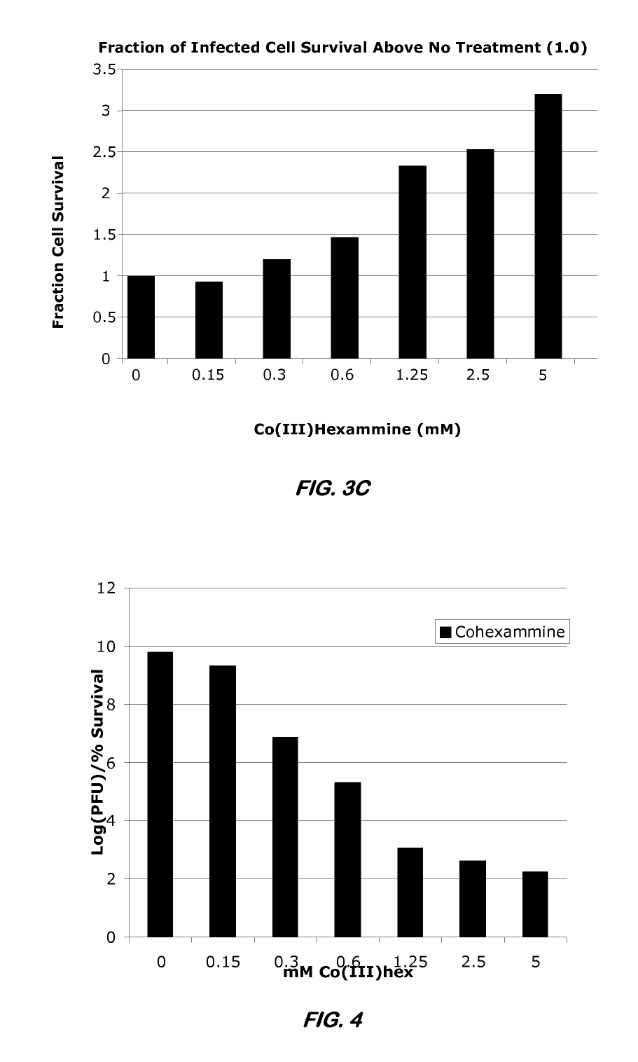

FIG. 3A is a graph illustrating a dose dependent decrease in the plaque counts, in log pfu, for each increasing concentration of Co(NH₃)₆ used at 48 hours. A maximal inhibition of virus replication (i.e., about a 2.5 log decrease in pfu over the control with no Co(NH₃)₆) was seen at about 2.5 mM Co(NH₃)₆. FIG. 3A shows that Co(NH₃)₆ decreases plaque formation by almost 2 to 3 log units. These same general trends were observed in at 24-hours post infection (data not provided).

In FIG. 3B, the degree of inhibition of plaque formation units measured in FIG. 3A is plotted as a function of Co(NH₃)₆ concentration. The 50% inhibitory concentration (IC₅₀) for Co(NH₃)₆ inhibition of Sindbis virus plaque formation was determined to be about 0.10±0.04 mM.

The reductions in viral pfu of these magnitudes in vitro suggest in vivo significance. Previous studies have shown that a one log unit decrease in viral load in murine brains correlates with survival of infected animals. However, FIG. 3A does not take into consideration a significant decrease in cell proliferation due to increased cytotoxicity of Co(NH₃)₆ at concentrations over 2.5 mM or even a slight decrease in cell proliferation at concentrations less than 2.5 mM, as illustrated in FIG. 1. Nor does FIG. 3A account for the decrease in cell proliferation due to the virus replication itself.

### EXAMPLE 6

Since the use of a CoHex complex of the present invention reduces but does not eliminate cell death due to the viral activity, the viability of BHK cells during Sindbis virus infection must also be determined as a function of the concentration of the CoHex complex. Because live cell counts will decrease with increasing CoHex complex due to cytotoxicity of the cells and virus replication, but increase due to the anti-viral protection of CoHex, the plaque counts may have decreased due to the fact that there were less live cells available to count. FIG. 3C is a graph depicting the fraction of cell survival of BHK cells during Sindbis viral infection as a function of Co(NH₃)₆ concentration at 48 hours after infection, in which cell survival is expressed as a “fraction cell survival” relative to Sindbis-infected cells that received no Co(NH₃)₆ treatments. The data of FIG. 3C was obtained using the CellTiter96 cell proliferation assay described above for the CoHex cytotoxicity data (i.e., with respect to FIG. 1) in which the cells were further infected with 5 plaque forming units per cell of Sindbis virus in DMEM containing 2% FBS in the absence or presence of various concentrations of Co(NH₃)₆, then assayed periodically.

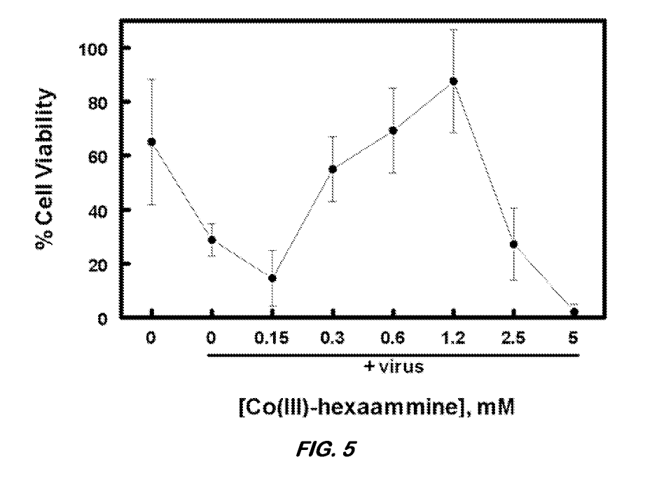

Therefore, the fraction cell survival data of FIG. 3C was combined with the log pfu data of FIG. 3A to generate a ratio, Log(PFU) per percent cell survival, which although not a physical measure, is a general model of viruses in surviving cells. FIG. 4 shows this ratio for the various concentrations of Co(NH₃)₆ used during the infection of the BHK cells. As illustrated in FIG. 4, there has been a significant decrease in viral infection for the surviving cells with an increase in concentration of Co(NH₃)₆.

### Cell Viability Study

### EXAMPLE 7

Additional cell viability studies were done using a combined assessment of cellular morphology via light microscopy, or visual inspection of cell viability, and plasma membrane integrity measurements using a Trypan blue dye-exclusion assay. Specifically, BHK cells were seeded to the wells of a 96-well plate (2×10⁴ cells/well) and cultured overnight in complete growth medium. The cells were then incubated with increasing concentrations of Co(NH₃)₆ for 6 hours prior to the addition of Sindbis virus (1×10⁵ pfu/well). After a 48 hour infection period, the cells were resuspended and an aliquot of cells (10 μL) was mixed with 90 μL 0.2% trypan blue. The number of viable cells was determined by counting with a hemocytometer.

FIG. 5 illustrates the results of this viable cell count as a function of Co(NH₃)₆ concentration. FIG. 5 reflects that after 48 hours, cell membrane viability was only about 65% for the uninfected control and was down to 30% with virus but no Co(NH₃)₆ added. The initial addition of 0.15 mM Co(NH₃)₆ further decreased viability, but higher concentrations revived cell viability up to 80% at 1.2 mM, beyond which point, survivability dropped drastically. Due to the large scatter in the range of data values, there is not a significant difference between the 65% uninfected and the 80% 1.2 mM Co(NH₃)₆ values.

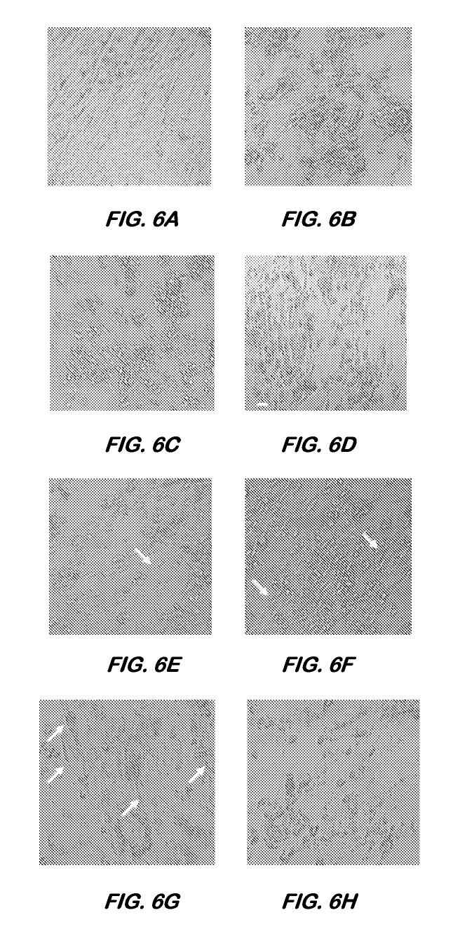

Also, a corresponding set of wells was identically prepared exclusively for observation using Trypan blue staining and light microscopy. FIGS. 6A-6H are light microscopy images showing cell morphology at various concentrations of Co(NH₃)₆ at 48 hours post infection following 6 hours of pretreatment with Co(NH₃)₆. Specifically, FIG. 6A shows healthy BHK cells in the absence of both Sindbis virus and Co(NH₃)₆. FIG. 6B shows BHK cells infected with the Sindbis virus but no Co(NH₃)₆. FIG. 6C shows BHK cells infected with the Sindbis virus and 0.15 mM Co(NH₃)₆. FIG. 6D shows BHK cells infected with the Sindbis virus and 0.3 mM Co(NH₃)₆. FIG. 6E shows BHK cells infected with the Sindbis virus and 0.6 mM Co(NH₃)₆. FIG. 6F shows BHK cells infected with the Sindbis virus and 1.2 mM Co(NH₃)₆. FIG. 6G shows BHK cells infected with the Sindbis virus and 2.5 mM Co(NH₃)₆. FIG. 6H shows BHK cells infected with the Sindbis virus and 5 mM Co(NH₃)₆. Viable cells remain attached to the tissue culture substrate and have an elongated, epithelial cell-like appearance. The arrows in FIGS. 6E-6G indicate examples of cells that have retained an elongated and adherent morphology. Non-viable cells are detached from the tissue culture substrate and are rounded. FIGS. 6B-6H illustrate that pronounced cell death is apparent in the presence of no Co(NH₃)₆ (FIG. 6B) and at lower concentrations of Co(NH₃)₆ (FIGS. 6C and 6D). However, viable cells appeared more pronounced at concentrations of 0.6 and 1.2 mM Co(NH₃)₆ (FIGS. 6E and 6F, respectively), again decreasing at 2.5 mM and 5.0 mM Co(NH₃)₆ (FIGS. 6G and 6H, respectively).

It is interesting to note that both the light microscopy results (FIGS. 6A-6H) and the cell viability count (FIG. 5) show similar results, and both suggest indicated that 1.2 mM concentration of Co(NH₃)₆ provides an optimal protection against Sindbis virus.

### Flow Cytometry-EGFP Reporter Assay

### EXAMPLE 8

Additional studies of antiviral activity were quantified by flow cytometry, a more sensitive fluorometric assay, to further validate the plaque assay and cell viability results. In these assays, Sindbis virus replication was assessed by monitoring viral structural protein synthesis using a recombinant Sindbis virus construct. In this construct, the gene encoding enhanced green fluorescent protein (EGFP) was placed under the control of the same promoter sequence that drives transcription of viral structural proteins. Thus, upon Sindbis virus replication, virally infected cells produce intracellular EGFP at levels that are proportional to the level of viral structural proteins.

BHK cells (1×10⁵) were plated into the wells of 24-well plates in 1 mL of complete media and incubated with increasing concentrations of Co(NH₃)₆ for 6 hours prior to infection with Sindbis virus at a multiplicity of infection (moi) of 5 pfu per cell. The infection was allowed to proceed for 48 hours. In preparation for analysis by flow cytometry, cells from each well were removed and pelleted, washed with PBS, and resuspended in 500 μL of PBS. One half of each sample was analyzed directly to determine the percentage of EGFP-positive cells (percent infected cells). The other half of each sample was incubated with 5 μM propidium iodide for 1 minute prior to the determination of the percentage of non-viable cells. In all cases, analysis was performed on 1×10⁴ cells.

BHK cells were pretreated with Co(NH₃)₆ for 6 hours prior to infection with Sindbis virus. After 48 hours post infection, both the percentage of Sindbis-infected cells (as evidenced by EGFP fluorescence) and the percentage of viable cells (as determined by the exclusion of propidium iodide (PI)) were determined as a function of Co(NH₃)₆ concentrations.

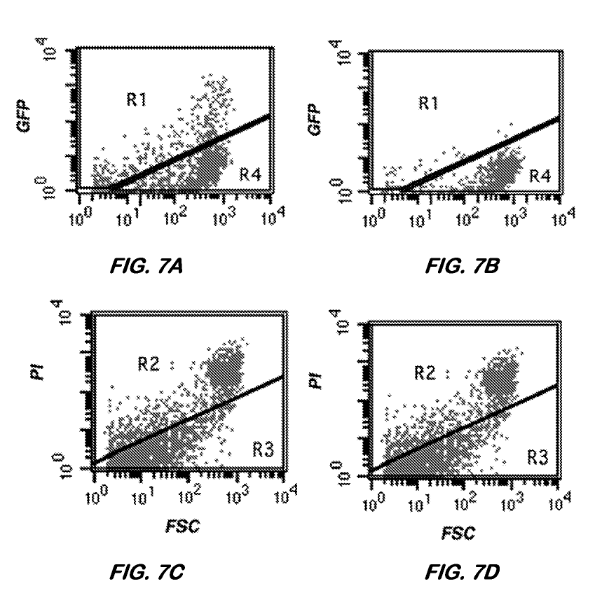

FIGS. 7A and 7B are representative raw data plots of simultaneous analysis of SV protein synthesis during SV infection in the absence of Co(NH₃)₆ (FIG. 7A) and in the presence of 0.15 mM Co(NH₃)₆ (FIG. 7B). The area marked ‘R1’ corresponds to EGFP-positive (SV-infected) cells. In the presence of Co(NH₃)₆, a distinct decrease in the percentage of EGFP-positive (i.e., Sindbis-infected) cells was apparent. FIGS. 7C and 7D are representative flow cytometry data for the viability of SV-infected BHK cells in the absence of Co(NH₃)₆ (FIG. 7C) and in the presence of 0.15 mM Co(NH₃)₆ (FIG. 7D). The area marked ‘R2’ corresponds to propidium iodide (PI)-positive (non-viable) cells. In all cases, the x-axis corresponds to forward scatter (FSC). Quantification of cell viability revealed that during Sindbis infection, relative to infected cells that were not treated with Co(NH₃)₆ (FIG. 7C, region R2), cells treated with 0.15 mM Co(NH₃)₆ exhibited a significantly lower percentage of PI-positive (non-viable) cells (FIG. 7D, region R2).

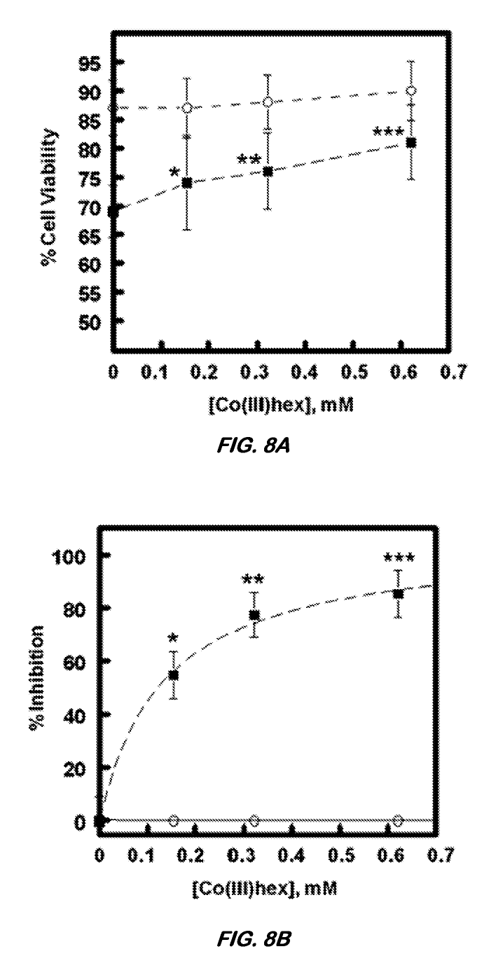

Data demonstrating the dose-dependent nature of the Co(NH₃)₆-mediated increase in cell viability and inhibition of EGFP expression (i.e. inhibition of SV replication) as a function of Co(NH₃)₆ concentrations are presented in FIGS. 8A and 8B, respectively. In FIG. 8A, data are shown for the dose-dependent increase in cell viability in SV-infected cells (solid square) as a function of Co(NH₃)₆ concentrations relative to control, uninfected cells (open circle). In FIG. 8B, data are shown for the dose-dependent inhibition of EGFP expression in SV-infected cells (solid square) as a function of Co(NH₃)₆ concentration compared to control, uninfected cells (open circle). In FIGS. 8A and 8B, asterisks on the plots for the SV-infected cells correspond to levels of significance relative to the untreated infected control (as determined by a two-tailed Student's t-test): *(p<0.05); **(p<0.005); ***(p<0.001). When the data in FIG. 8B were fitted to a standard dose-response curve using a one-site dose response logistic curve fit function, an IC₅₀ of 0.13±0.04 mM was determined. This value agrees well with the IC₅₀ value determined by the plaque assay above, (0.10±0.04 mM).

A selectivity ratio may be calculated by comparing CC₅₀ to IC₅₀: CC₅₀/IC₅₀=about 25. This selectivity ratio measures the selectivity of Co(NH₃)₆ for Sindbis virus over BHK cells. Thus, Co(NH₃)₆ is about 25 times more selective for the virus than for the host cell.

### Timing of CoHex Administration on Antiviral Activity

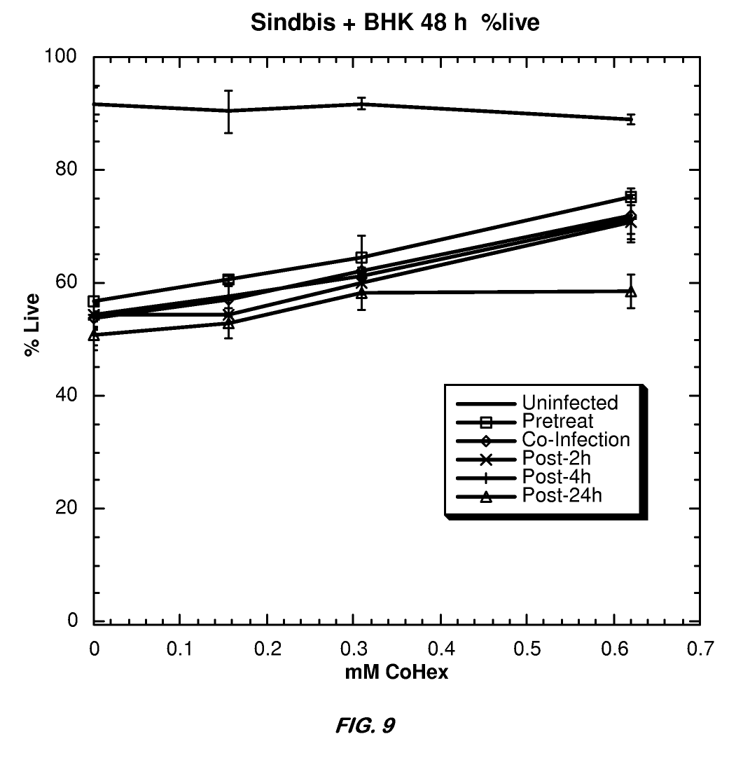

The flow cytometry assay discussed above was repeated with various concentrations of Co(NH₃)₆ administered as a pretreatment to Sindbis viral infection, as co-infection and at 2, 4 and 24 hours post-infection. FIG. 9 is a chart illustrating survival rates for BHK cells for the various concentrations of Co(NH₃)₆ for each administration timing. As seen in FIG. 9, timing of the Co(NH₃)₆ treatment has very little effect on the survivability of the cells. As such, Co(NH₃)₆ is an effective antiviral agent whether administered prior to viral infection, contemporaneously with viral infection, or after viral infection. However, it appears that early treatment of Co(NH₃)₆ generally increases the percentage of cell survival.

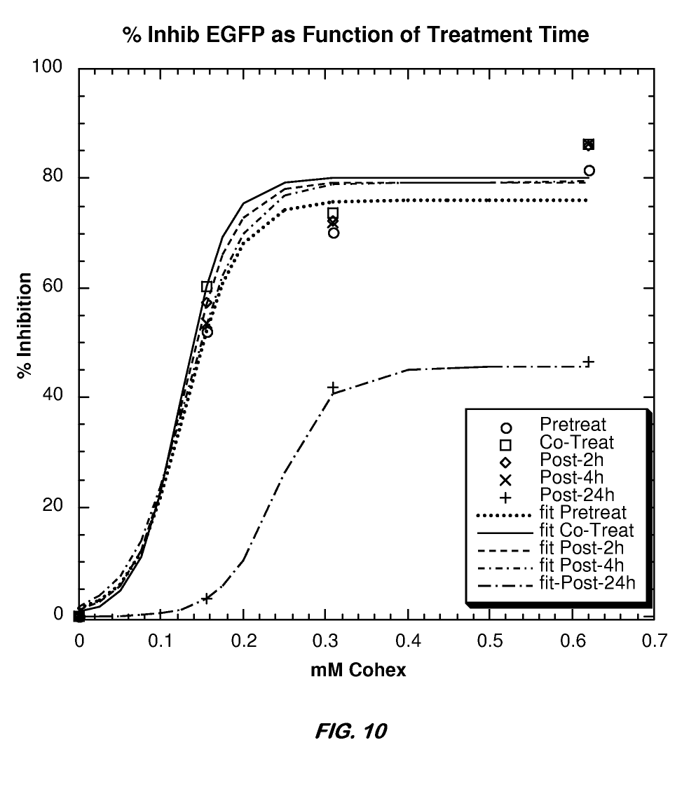

Similarly, FIG. 10 is a chart illustrating the effects of timing of Co(NH₃)₆ administration on EGFP expression by Sindbis virus in BHK cells. The chart measures percent inhibition of EGFP expression as a function of Co(NH₃)₆ concentration. As illustrated, Co(NH₃)₆ is equally effective when administered as a pretreatment, co-infection or early post-treatment. Even at 24 hours post-treatment, inhibition of EGFP expression is apparent, but not apparently as significantly as with earlier treatment.

### Antiviral Activity of the CoHex Complexes—Adenovirus

### EXAMPLE 10

Another example of antiviral activity is an in vitro model of a double stranded DNA (dsDNA) virus infection, Adenovirus. As with Sindbis virus, effective antiviral activity was demonstrated with Adenovirus in A549 cells. The same flow cytometry procedure discussed above for Sindbis viral infection in BHK cells was used for Adenovirus viral infection in A549 cells. In other words, the cells were incubated with 5 μM propidium iodide for 1 minute prior to the determination of the percentage of non-viable cells. The percentage of viable cells (as determined by the exclusion of propidium iodide (PI)) were determined as a function of Co(NH₃)₆ concentration.

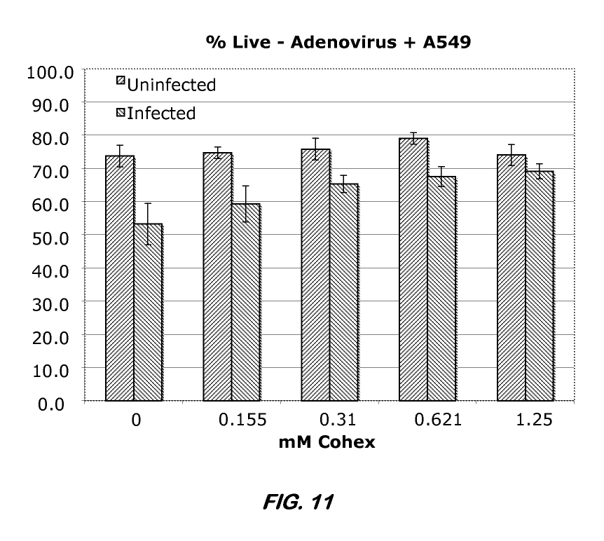

FIG. 11 illustrates survival rates for A549 cells uninfected and infected with Adenovirus assayed at 48 hours post infection with increasing concentration of Co(NH₃)₆ administered as a 6 hour pretreatment. As such, the presence of increasing concentration of Co(NH₃)₆, up to 0.0621 mM Co(NH₃)₆, demonstrates an increase in survival rate of A549 cells. A continued increase in survivability of A549 cells was found at a concentration of 1.25 mM Co(NH₃)₆, despite the fact that the toxicity began to take effect at this high concentration as evidenced by the reduction in cell survivability of the uninfected cells at 1.25 mM Co(NH₃)₆ (FIG. 1).

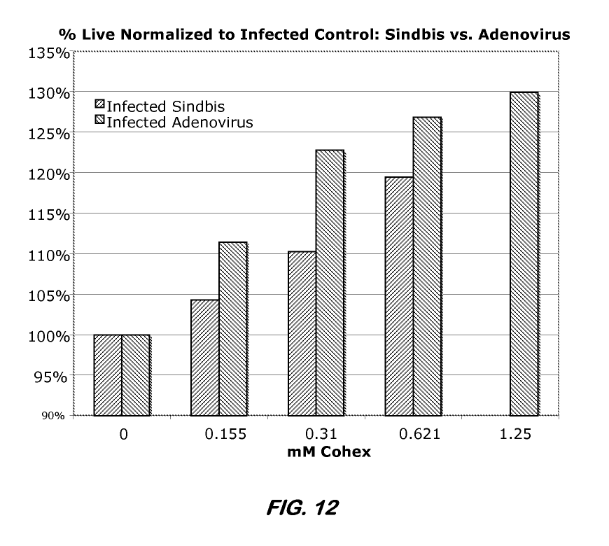

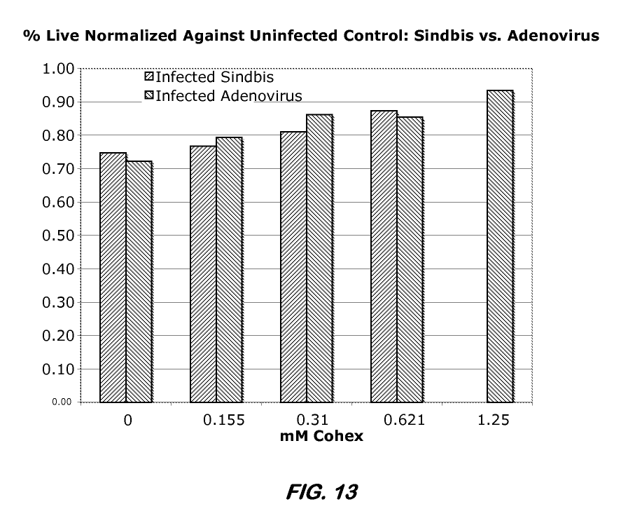

As illustrated in FIGS. 12 and 13, the normalized survivability rates of Co(NH₃)₆ treated cells is similar whether the viral infections are Adenovirus infections or Sindbis virus infections. In both cases, Co(NH₃)₆ increases cell survivability of virally infected cells.

### Sequence Specific CoHex System

To be specific for a particular protein, the CoHex system of the present invention will need to possess an ability to recognize sequences of nucleotides that code for that protein. Thus, a full sequence-recognizing, artificial nuclease can be thought to consist of a very tight binding component and a sequence-recognition nucleotides component.

Thus, the present invention also includes the addition of nucleotide-binding groups, such as hybridization-capable oligonucleotides, or other nucleotide-binding groups (e.g., peptide nucleic acids, modified nucleic acids, triple-helix formers, or nucleotide-binding proteins and drugs), which are attached to functionalized CoHex complexes. The resulting system will be able to identify and bind to specific genes in order to silence transcription/translation of specific genes and/or attack specific viruses. Further, the attachment of a hybridizing oligonucleotide sequence to a CoHex complex may enable the CoHex complex to recognize nucleotide sequences that are longer than those typically used for RNAi applications. Therefore, CoHex complexes can potentially be used for both non-sequence-specific and sequence-specific inhibition of transcription/translation of viral proteins, as well as for other molecular biology tasks. The molecular recognition potential of the chemical nucleases, furthermore, means that these chemical nucleases can potentially be used in a novel approach to interdict whole classes of organisms (e.g., the filoviruses).

### EXAMPLE 10

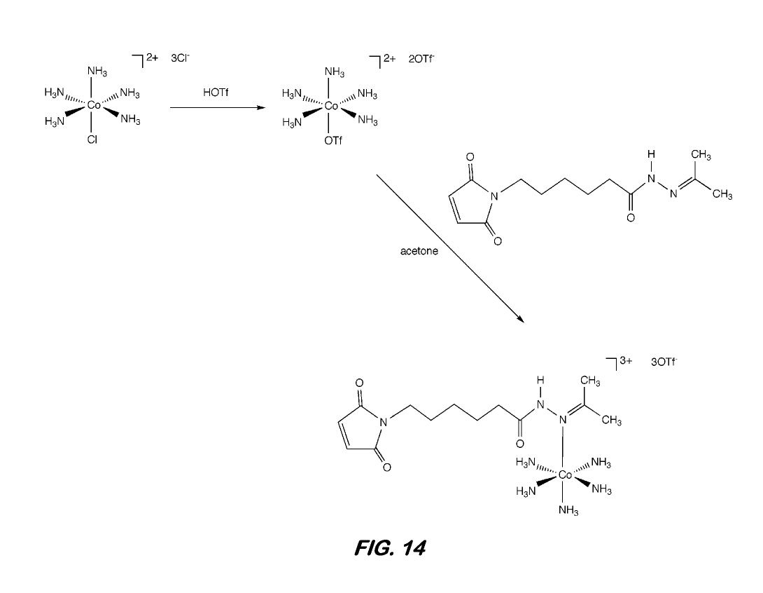

Although a variety of nucleotide-binding groups that identify and bind to a particular sequence may be added to the CoHex complexes of the present invention, one method for doing so includes starting with a copentammine salt, such as a chloropentammine cobalt(III) salt and mono-substituting the non-ammine coordination site with a functional group, which can bind to the CoHex complex. The functional group can subsequently be attached to an oligonucleotide or oligonucleotide sequence designed to identify and bind with specific viral DNA or RNA. For example, FIG. 14 illustrates a schematic method for synthesizing a CoHex complex having a mono-substituted functional group in the R₁ position of Structure III, as shown in Structure VI below. In Structure VI and in FIG. 14, “OTf” refers to a triflate anion, CF₃SO₃⁻.

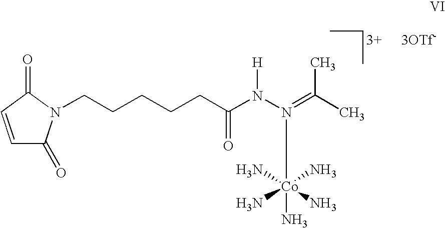

Structure VI was synthesized by mono-substitution of chloropentamminecobalt(III) chloride with trifluoromethanesulfonic acid. The brick red product was collected, then treated with (N-[ε-Maleimidocaproic acid]hydrazide) in dry acetone and stirred for 24 hrs. The pink solution was washed with dichloromethane to remove any organic impurities and then dried to give Structure VI as a pink powder. Structure VI, for example, may be particularly useful to bond with oligonucleotide or oligonucleotide sequences having a thiol functional group.

One skilled in the art can appreciate synthesis of CoHex complexes with a variety of functional groups as would be appropriate for a particular application either using the above described method or an appropriate alternative method. Such a CoHex complex would be capable of targeting and inhibiting translation of a particular sequence or virus.

While the invention has been particularly shown and described with reference to preferred embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents. Additionally, all references cited herein, including issued U.S. patents, or any other references, are each entirely incorporated by reference herein, including all data, tables, figures, and text presented in the cited references. Also, it is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

What is claimed as new and desired to be protected by Letters Patent of the United States is:

1. A method of prophylaxis against viral infection of a cell, comprising:
administering to a cell an antiviral composition having the structure of Structure III, 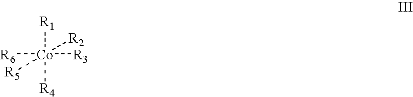
wherein each of R₁, R₂, R₃, R₄, R₅ and R₆ is the same or different and includes a N-based ligand donor atom selected from the group consisting of ammonia, primary amine or secondary amine, or salt thereof, so as to thereby provide prophylaxis against infection of the cell by a virus.

2. The method of claim 1, wherein each of R₁, R₂, R₃, R₄, R₅ and R₆ is an ammonia ligand.

3. The method of claim 1, wherein the antiviral composition is a Co(NH₃)₆ salt.

4. The method of claim 1, wherein R₁ includes a nucleotide-binding group.

5. The method of claim 1, wherein the administration of the antiviral composition causes at least a two log pfu reduction in viral activity over no antiviral composition added.

6. The method of claim 1, wherein said viral infection is from a +ssRNA virus.

7. The method of claim 1, wherein said viral infection is from a dsDNA virus.

8. The method of claim 1, wherein the antiviral composition is less toxic than a conventional cis-Platin complex.

9. The method of claim 1, wherein the antiviral composition has a high positively charged density.

10. The method of claim 1, wherein the antiviral composition does not hydrolyze nucleotides.

11. The method of claim 1, wherein the antiviral composition is administered prior to the viral infection.

12. The method of claim 1 wherein the antiviral composition is administered contemporaneously with the viral infection.

13. The method of claim 1, wherein the antiviral composition is administered subsequent to the viral infection.

14. A method of treating a subject infected with a virus comprising:
administering to the subject an antiviral composition comprising an antiviral effective amount of a compound having the structure of Structure III, 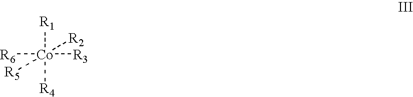
wherein each of R₁, R₂, R₃, R₄, R₅ and R₆ is the same or different and includes a N-based ligand donor atom selected from the group consisting of ammonia, primary amine or secondary amine, or salt thereof, so as to thereby treat the subject infected by the virus.

15. The method of claim 14, wherein R₁ includes a nucleotide-binding group.

16. A method of prophylaxis against viral infection of a subject, comprising:
administering to a cell an antiviral composition having the structure of Structure III, 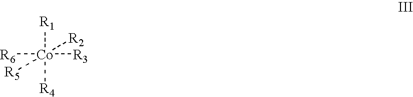
wherein each of R₁, R₂, R₃, R₄, R₅ and R₆ is the same or different and includes a N-based ligand donor atom selected from the group consisting of ammonia, primary amine or secondary amine, or salt thereof, so as to thereby provide prophylaxis against infection of the subject by the virus.

17. The method of claim 16, wherein R₁ includes a nucleotide-binding group.

18. A method of preparing an antiviral agent comprising:
providing a cobalt pentammine salt having a non-amine coordination site;
mono-substituting the non-amine coordination site with a functional group incorporating a strong coordinator atom to cobalt to form a CoHex structure of Structure III, 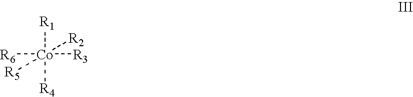
wherein each of R₂, R₃, R₄, R₅ and R₆ is the same or different and includes a N-based ligand donor atom selected from the group consisting of ammonia, primary amine or secondary amine and R₁ incorporates the functional group having the strong coordinator atom coordinated with the cobalt atom or a salt thereof.

19. The method of claim 18, wherein the strong coordinator atom is a nitrogen atom.

20. The method of claim 18, further comprising the step of binding the functional group to at least one oligonucleotide.
